(19) United States
(12) Patent Application Publication
(10) Pub. No.: US 2007/0092488 A1
(43) Pub. Date: Apr. 26, 2007

(54) Methods of treating idiopathic pulmonary fibrosis

(75) Inventors: Robert M. Strieter, Sherman Oaks, CA (US); Karen Starko, Hillsborough, CA (US)

Correspondence Address: FOLEY & LARDNER LLP, P.O. BOX 80278, SAN DIEGO, CA, 92138-0278, US

(73) Assignee: Intermune Inc., Brisbane, CA (US)

(21) Appl. No.: 10/557,110

(22) PCT Filed: May 14, 2004
(86) PCT No.: PCT/US04/15174

§ 371 Date: Sep. 5, 2006

Related U.S. Application Data
(60) Provisional application No. 60/471,199, filed on May 16, 2003.

(51) Int. Cl. A61K 38/21; C12Q 1/68; A61K 38/22

(52) U.S. Cl. 424/85.6; 435/6; 514/12

(57) ABSTRACT

The present invention provides methods of treating idiopathic pulmonary fibrosis (IPF); methods of increasing survival time in an individual with IPF; and methods of reducing risk of death in an individual with IPF. The methods generally involve administering a therapeutically effective amount of IFN-γ to an individual with IPF.

INTERMUNE, INC., applicant, a United States national and resident, and Robert M. Strieter and Karen M. Starko; inventors/applicants, United States nationals and residents; are filing this application as a PCT application claiming priority to U.S. Provisional Patent Application No. 60/471,199 filed 16 May 2003.

### BACKGROUND OF THE INVENTION

Pulmonary fibrosis can be caused by a number of different conditions, including sarcoidosis, hypersensitivity pneumonitis, collagen vascular disease, and inhalant exposure. The diagnosis of these conditions can usually be made by careful history, physical examination, chest radiography, including a high resolution computer tomographic scan (HRCT), and open lung or transbronchial biopsies. However, in a significant number of patients, no underlying cause for the pulmonary fibrosis can be found. These conditions of unknown etiology have been termed idiopathic interstitial pneumonias. Histologic examination of tissue obtained at open lung biopsy allows classification of these patients into several categories, including Usual Interstitial Pneumonia (UIP), Desquamative Interstitial Pneumonia (DIP), and Non-Specific Interstitial Pneumonia (NSIP).

The logic of dividing idiopathic interstitial pneumonias into these categories is based not only on histology, but also on the different response to therapy and prognosis for these different entities. DIP is associated with smoking and the prognosis is good, with more than 70% of these patients responding to treatment with corticosteroids. NSIP patients are also frequently responsive to steroids and prognosis is good, with 50% of patients surviving to 15 years. In contrast, the UIP histologic pattern is associated with a poor response to therapy and a poor prognosis, with survival of only 3-5 years.

Idiopathic pulmonary fibrosis (IPF) is the most common form of idiopathic interstitial pneumonia and is characterized by the UIP pattern on histology. IPF has an insidious onset, but once symptoms appear, there is a relentless deterioration of pulmonary function and 50% mortality within 3-5 years after diagnosis. The mean age of onset is 60-65 and males are affected approximately twice as often as females. Prevalence estimates are 13.2-20.2 per 100,000. The annual incidence is estimated to be 7.4-10.7 per 100,000 new cases per year. See, for example, American Thoracic Society (ATS), and the European Respiratory Society (ERS), 2000, Am J Respir Crit Care Med. 161(2 Pt 1):646-64, “Idiopathic pulmonary fibrosis: diagnosis and treatment. International consensus statement.”; and American Thoracic Society/European Respiratory Society, June 2001, Am J Respir Crit Care Med 165(2):277-304, “International Multidisciplinary Consensus Classification of the Idiopathic Interstitial Pneumonias.” This joint statement of the American Thoracic Society (ATS), and the European Respiratory Society (ERS) was adopted by the ATS board of directors, June 2001 and by the ERS Executive Committee, June 2001.

Published evidence suggests that less than 20% of patients with IPF respond to steroids. In patients who have failed treatment with steroids, cytotoxic drugs such as azathioprine or cyclophosphamide are sometimes added to the steroid treatment. However, a large number of studies have shown little or no benefit of these drugs. There are currently no drugs approved for treatment of IPF.

The primary histopathologic finding of IPF is that of usual interstitial pneumonia with temporal heterogeneity of alternating zones of interstitial fibrosis with fibroblastic foci (i.e., newer fibrosis), inflammation, honeycomb changes (i.e., older fibrosis), and normal lung architecture (i.e., no evidence of fibrosis). In conjunction with the fibrotic process there is evidence for aberrant vascular remodeling. The pathogenesis of IPF is complex. A specific cause is unknown, and may be related to a number of various infectious agents, environmental exposure, and toxins in a genetically susceptible individual. See, for example, Katzenstein et al., 1998, Am J Respir Crit Care Med 157(4 Pt 1):1301-15, “Idiopathic pulmonary fibrosis: clinical relevance of pathologic classification.” and Keane, et al., 1997, J Immunol 159(3):1437-43, “The CXC chemokines, IL-8 and IP-10, regulate angiogenic activity in idiopathic pulmonary fibrosis.”

There is a need for methods of treating IPF. The present invention addresses this need.

### SUMMARY OF THE INVENTION

The present invention provides methods of treating idiopathic pulmonary fibrosis (IPF); methods of increasing survival time in an individual with IPF; and methods of reducing risk of death in an individual with IPF. The methods generally involve administering a therapeutically effective amount of IFN-γ with an IFN-γ-inducible CXCR₃ cytokine such as I-TAC/CXCL11, and/or an antagonist of a CXCL cytokine such as ENA-78/CXCL5. In the method invention, IFN-γ, I-TAC/CXCL11, an antagonist of ENA-78/CXCL5, or combination thereof may be co-administered with an antagonist of IL-4, an antagonist of PDGF-B, or a combination thereof, as well as co-administered with pirfenidone or a pirfenidone analog.

The invention further provides methods for evaluating IPF patient response to IFN-γ therapy by comparing post-treatment levels of I-TAC/CXCL11 and/or ENA-78/CXCL5 with control levels, and correlating a relative increase in I-TAC/CXCL11 and/or decrease in ENA-78/CXCL5 with patient response to IFN-γ.

The invention further provides methods for evaluating patient response to treatment with IFN-γ by analyzing expression of IL4, PDGF-B, or both, in an IFN-γ-treated patient. The methods further provide correlating decreased IL-4 expression, decreased PDGF-B expression, or both, as compared to a control expression, with patient response to IFN-γ treatment.

The invention further provides methods for evaluating patient response to treatment with IFN-γ by analyzing expression of elastin, procollagen III, or both, in an IFN-γ-treated patient. The methods further provide correlating decreased elastin expression, decreased procollagen III expression, or both, as compared to a control expression, with patient response to IFN-γ treatment.

### BRIEF DESCRIPTION OF THE DRAWINGS

FIG. 1 depicts survival probability in patients, having less than 55% of predicted forced vital capacity, treated with IFN-γ 1b or placebo.

FIG. 2 depicts the survival probability in patients, having at least 55% predicted forced vital capacity, treated with IFN-γ 1b or placebo.

FIG. 3 is a diagrammatic representation of potential factors contributing to IPF and expected responses of biomarkers to IFN-γ 1b therapy based on preclinical studies.

FIG. 4 is a graph showing the relative expression of biomarker mRNA in IFN-γ treated patients as compared with placebo controls.

FIG. 5 is a graph showing I-TAC/CXCL11 and ENA-78/CXCL5 protein levels in BAL fluids obtained from IFN-γ treated patients as compared with placebo controls.

FIG. 6 is a graph showing I-TAC/CXCL11 baseline and post-treatment protein levels in the plasma of IFN-γ treated patients as compared with placebo controls.

FIG. 7 is a graph demonstrating I-TAC/CXCL11 treatment attenuates bleomycin-induced pulmonary fibrosis in mice, as determined by reduction in total, soluble collagen.

FIG. 8 is a photomicrograph of lung tissue of mice treated with bleomycin to induce pulmonary fibrosis, and showing preservation of lung architecture in the lung tissue of mice treated with I-TAC/CXCL11.

FIG. 9 is a graph showing elevated ENA-78/CXCL5 in lung tissue of IPF patients as compared with normal lung tissue.

FIG. 10 is a panel of photomicrographs showing reduced vascular remodeling in anti-ENA-78/CXCL5-treated lung tissue as compared with untreated IPF lung tissue.

### Definitions

The term “antibody” is used in the broadest sense and specifically includes recombinant antibodies, monoclonal antibodies (including full length monoclonal antibodies), polyclonal antibodies, multispecific antibodies (e.g., bispecific antibodies), humanized antibodies, and antibody fragments so long as they exhibit the desired biological activity.

“Antibody fragments,” as defined for the purpose of the present invention, comprise a portion of an intact antibody, generally including the antigen binding or variable region of the intact antibody. Examples of antibody fragments include linear antibodies; single-chain antibody molecules; diabodies, linear antibodies (Zapata et al., Protein Eng. 8(10): 1057 (1995)) and multispecific antibodies formed from antibody fragments. Included within the definition of “antibody fragments” are Fv, Fv′, Fab, Fab′, and F(ab′)₂ fragments.

Agents that “reduce or avoid dysregulated angiogenesis” include those that induce or establish angiostasis, or prevent or reduce the processes of new or abnormal blood vessel growth (neovascularization).

The term “antagonist” is used in the broadest sense, and includes any molecule that partially or fully blocks, inhibits, or neutralizes a biological activity of a native biologically active molecule. An antibody or antibody fragment possessing antagonist activity is included within the scope of the term “antagonist”.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

The terms “individual,” “host,” “subject,” and “patient,” used interchangeably herein, refer to a mammal, particularly a human.

Improvement refers herein to an increase of at least 10% in the percent predicted FVC from baseline value.

An “effective” amount of an agent is meant to mean an amount of a therapeutic agent, or a rate of delivery of a therapeutic agent, effective to facilitate a desired therapeutic effect. The precise desired therapeutic effect will vary according to the condition to be treated, the formulation to be administered, and a variety of other factors that are appreciated by those of ordinary skill in the art.

A “fibrotic condition,” “fibrotic disease” and “fibrotic disorder” are used interchangeably to refer to a condition, disease or disorder that is characterized by progressive accumulation of fibrous tissue. Fibrotic disorders include, but are not limited to, pulmonary fibrosis, including idiopathic pulmonary fibrosis (IPF) and pulmonary fibrosis from a known etiology, liver fibrosis, and renal fibrosis. Other exemplary fibrotic conditions include musculoskeletal fibrosis, cardiac fibrosis, post-surgical adhesions, scleroderma, glaucoma, and skin lesions such as keloids.

A “specific pirfenidone analog,” and all grammatical variants thereof, refers to, and is limited to, each and every pirfenidone analog shown in Table 1.

“Synergistically effective” is used to indicate that the combination of two or more agents is more effective in therapeutic or prophylactic treatment than could be predicted or expected from a merely additive combination of the agents.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a method” includes a plurality of such methods and reference to “an IFN-γ dose” includes reference to one or more doses and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods of treating idiopathic pulmonary fibrosis (IPF); methods of increasing survival time in an individual with IPF; and methods of reducing risk of death in an individual with IPF. The methods generally involve administering a therapeutically effective amount of IFN-γ, I-TAC/CXCL11, antagonist of ENA-78/CXCL5, an antagonist of IL-4, an antagonist of PGDF-B, or a combination thereof, to an individual with IPF.

Idiopathic Pulmonary Fibrosis

IPF is a disease of unknown etiology characterized by the accumulation of neutrophils and mononuclear cells, followed by the progressive deposition of collagen within the interstitium and subsequent destruction of lung airspaces. See, e.g., Vaillant et al. (1996) Monaldi. Arch. Chest Dis. 51: 145; Phan, (1995) Thorax 50: 415. Activated alveolar macrophages and neutrophils are believed to play a significant role in the pathogenesis of the characteristic inflammatory lung lesions found in patients with IPF. Increasing scientific evidence points to the importance of neutrophils in the pathogenesis of IPF. Neutrophils are potent immune effector cells, and can release oxygen radicals, complement fragments, arachidonic acid metabolites, proteolytic enzymes, and various cytokines, all of which may inflict lung injury.

The pathology of IPF additionally demonstrates features of dysregulated and abnormal repair with exaggerated angiogenesis, fibroproliferation, and deposition of extracellular matrix, leading to progressive fibrosis and loss of lung function.

Interferon-Gamma

IFN-γ is a pleiotropic cytokine with antimicrobial, antifibrotic/antiproliferative, and immunomodulator properties. IFN-γ1b (Actimmune®; human interferon) is a single-chain polypeptide of 140 amino acids. It is made recombinantly in E. coli and is unglycosylated. Rinderknecht et al., 1984, J. Biol. Chem. 259:6790-6797. The nucleic acid sequences encoding IFN-γ polypeptides may be accessed from public databases, e.g. Genbank, journal publications, etc. While various mammalian IFN-γ polypeptides are of interest, for the treatment of human disease, generally the human protein will be used. Human IFN-γ coding sequence may be found in Genbank, accession numbers X13274; V00543; and NM_{—}000619. The corresponding genomic sequence may be found in Genbank, accession numbers J00219; M37265; and V00536. See, for example. Gray et al., 1982, Nature 295:501 (Genbank X13274); and Rinderknecht et al., 1984 J. Biol. Chem. 259:6790.

IFN-γ binds to Type I interferon receptor, a cell surface receptor that consists of two transmembrane subunits, IFN-alphaR1 and IFN-alphaR2, which may be present in different forms. Roisman et al., 2001, P.N.A.S., 98:13231-13236; Petska, J., 1997, Semin. Oncol., 24:9-40; Yan et al., 1996, Mol. Cell Biol., 16:2074-2082; Novick et al., 1994, Cell, 77:391-400. The binding of a Type I interferon receptor agonist to a Type I interferon receptor activates multiple intracellular cascades leading to the synthesis of proteins that mediate antiviral, growth inhibitory, and immunomodulatory responses. Brierley and Fish, 2002, J. Interferon Cytokine Res., 22:835-845.

IFN-γ1b (Actimmune®; human interferon) is a single-chain polypeptide of 140 amino acids. It is made recombinantly in E. coli and is unglycosylated. Rinderknecht et al. (1984) J. Biol. Chem. 259:6790-6797. Recombinant IFN-γ as discussed in U.S. Pat. No. 6,497,871 is also suitable for use herein.

The IFN-γ to be used in the methods of the present invention may be any of natural IFN-γs, recombinant IFN-γs and the derivatives thereof so far as they have an IFN-γ activity, particularly human IFN-γ activity. Human IFN-γ exhibits the antiviral and anti-proliferative properties characteristic of the interferons, as well as a number of other immunomodulatory activities, as is known in the art. Although IFN-γ is based on the sequences as provided above, the production of the protein and proteolytic processing can result in processing variants thereof. The unprocessed sequence provided by Gray et al., supra, consists of 166 amino acids (aa). Although the recombinant IFN-γ produced in E. coli was originally believed to be 146 amino acids, (commencing at amino acid 20) it was subsequently found that native human IFN-γ is cleaved after residue 23, to produce a 143 aa protein, or 144 aa if the terminal methionine is present, as required for expression in bacteria. During purification, the mature protein can additionally be cleaved at the C terminus after reside 162 (referring to the Gray et al. sequence), resulting in a protein of 139 amino acids, or 140 amino acids if the initial methionine is present, e.g. if required for bacterial expression. The N-terminal methionine is an artifact encoded by the mRNA translational “start” signal AUG that, in the particular case of E. coli expression is not processed away. In other microbial systems or eukaryotic expression systems, methionine may be removed.

For use in the subject methods, any of the native IFN-γ peptides, modifications and variants thereof, or a combination of one or more peptides may be used. IFN-γ peptides of interest include fragments, and can be variously truncated at the carboxyl terminus relative to the full sequence. Such fragments continue to exhibit the characteristic properties of human gamma interferon, so long as amino acids 24 to about 149 (numbering from the residues of the unprocessed polypeptide) are present. Extraneous sequences can be substituted for the amino acid sequence following amino acid 155 without loss of activity. See, for example, U.S. Pat. No. 5,690,925. Native IFN-γ moieties include molecules variously extending from amino acid residues 24-150; 24-151, 24-152; 24-153, 24-155; and 24-157. Any of these variants, and other variants known in the art and having IFN-γ activity, may be used in the present methods.

The sequence of the IFN-γ polypeptide may be altered in various ways known in the art to generate targeted changes in sequence. A variant polypeptide will usually be substantially similar to the sequences provided herein, i.e., will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. In one embodiment, the invention contemplates the use of IFN-γ variants with one or more non-naturally occurring glycosylation and/or pegylation sites that are engineered to provide glycosyl- and/or PEG-derivatized polypeptides with reduced serum clearance, such as the IFN-γ polypeptide variants described in International Patent Publication No. WO 01/36001. Also included are modifications of glycosylation, e.g., those made by modifying the glycosylation patterns of a polypeptide during its synthesis and processing or in further processing steps; e.g., by exposing the polypeptide to enzymes that affect glycosylation, such as mammalian glycosylating or deglycosylating enzymes. Also embraced are sequences that have phosphorylated amino acid residues, e.g., phosphotyrosine, phosphoserine, or phosphothreonine.

Included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g., D-amino acids or non-naturally occurring synthetic amino acids. The protein may be pegylated to enhance stability.

Despite the positive results from treatment of IPF with IFN-γ, some patients are unresponsive to the treatment. It would be useful to have a method for determining whether a patient with IPF responds to IFN-γ therapy.

Biomarkers

Some embodiments of the invention utilize biomarkers to determine patient response to IFN-γ. IFN-γ is known to induce multiple biological factors in vivo. Biomarkers that are regulated by IFN-γ can be classified into two, broad groups: those that are upregulated by IFN-γ treatment, and those that are downregulated by IFN-γ treatment. The present invention comprises in part the surprising discovery that I-TAC/CXCL11 is markedly upregulated by IFN-γ treatment, while ENA-78/CXCL5 is markedly downregulated by IFN-γ treatment. Failure of IFN-γ therapy to upregulate or downregulate an IFN-γ-regulated biomarker can indicate that the patient is not responding to IFN-γ therapy. Therefore, the present invention relates in part to the discovery that the measurement of factors that are regulated by IFN-γ are useful to determine whether a patient is responsive to IFN-γ therapy. Correlations between IFN-γ administration and increased or decreased expression of certain of an IFN-γ regulated biomarker relative to baseline expression can indicate whether a patient responds to IFN-γ treatment, and can be used to monitor patient therapy.

IFN-γ regulated biomarkers are analyzed, for example, prior to and post-IFN-γ therapy. Comparison of the pre and post levels of the biomarkers can indicate if a patient is responding to the treatment. In addition, comparison of the relative biomarker levels of two or more post-treatment samples taken in temporal sequence can indicate if the patient continues to respond, or if the therapy should be discontinued, adjusted in dose, and the like.

Various biomarkers useful in the invention are described more fully below.

CXC Chemokines

The existence of neovascularization in IPF patients has resulted in attention to the role CXC chemokines may play in IPF pathogenesis, since this subset of the chemokine family has been found to exert disparate effects in regulating angiogenesis. (Strieter et al., 1995, J. Biol. Chem. 270:27348)

Chemokines are a superfamily of cytokines that play significant roles in inflammatory and immune responses due mostly to their chemotactic activities towards various leukocyte subsets. (Widney et al., 2000, J. Immun. 6322) CXC is one of four chemokine families, the others being CC, C, and CX₃C, each of which possesses a different number and arrangement of conserved cysteine motifs. The CXC motif is defined by the presence of one amino acid between the first two highly conserved cysteines in the motif. The CXC family itself is divided into ELR and non-ELR chemokines, wherein ELR is a Glu-Leu-Arg tripeptide sequence adjacent to the CXC motif.

An ELR chemokine of particular interest is ENA-78/CXCL5 (epithelial cell-derived neutrophil-activating peptide-78/CXCL5), a potent neutrophil chemoattractant. Non-ELR chemokines of interest include IFN-inducible protein 10 (IP10/CXCL10), monokine induced by IFN-γ (MIG/CXCL9), and IFN-inducible T cell α chemoattractant (I-TAC/CXCL11). The biological activity of chemokines depends upon their interactions with G protein-coupled receptors on the surface of target cells; I-TAC/CXCL11, IP10CXCL10, and MIG/CXCL9 all bind to the chemokine receptor CXC chemokine receptor 3 (CXCR3).

It is a feature of the present invention that IFN-γ has been found to induce changes in the expression of members of the CXC family relative to baseline. Therefore, these chemokines can be useful as IFN-γ regulated biomarkers in methods of the invention directed towards determining a patients's response to IFN-γ therapy. Additionally, the role of non-ELR CXC chemokines in neutrophil activation and the role of ELR CXC chemokines in angiogenesis, two processes implicated in IPF pathogenesis, render them useful as agents for the treatment of IPF, or as targets for antagonists in the treatment of IPF. Specifically, IP10/CXCL10 and MIG/CXCL9, have been shown to possess angiostatic activity (Strieter et al., U.S. Pat. No. 5,871,723), while ENA-78/CXCL5, as a potent activator of neutrophil activity, is suspected of playing a significant role in acute and/or chronic inflammation See, e.g., Walz et al., 1991, J. Exp. Med. 174:1355; Goodman et al., 1996, Am. J. Respir. Crit. Care Med. 154: 602.

ENA-78/CXCL5 has been shown to be an important regulator of angiogenic activity in IPF. Keane et al., 2001, Am J. Resp. Crit Care Med. 164(12):2239. Therefore, antagonists of ENA-78/CXCL5 can be useful in methods of the invention for treatment of patients with IPF. Exemplary members of the CXC family of chemolines are briefly described below.

I-TAC/CXCL11

The nucleic acid sequences encoding I-TAC/CXCL11 polypeptides may be accessed from public databases, including Genbank, and journal publications. While various mammalian I-TAC polypeptides are of interest, for the treatment of human disease, generally the human protein will be used. Human I-TAC/CXCL11 genomic sequence may be found in Genbank, accession number AF030514. See, for example, Cole et al. 1998,J. Exp. Med. 187 (No. 12): 2009.

The predicted, mature product of the I-TAC/CXCL11 coding sequence is a polypeptide of 72 amino acids. I-TAC/CXCL11 (interferon-inducible T cell alpha chemoattractant) is named for it potent chemoattractant activity for interleukin (IL)-2-activated T cells.

A feature of the present invention is the discovery that I-TAC/CXCL11 is markedly upregulated in IPF patients after IFN-γ administration, and that administration of I-TAC/CXCL11 has been shown attenuate fibrosis in a murine animal model. See Example 3 below.

ENA-78/CXCL5

Epithelial neutrophil-activating peptide 78 (ENA-78/CXCL5), like I-TAC/CXCL11, is a member of the CXC chemokine family, but belongs to the ELR-containing CXC subgroup. The nucleic acid sequences encoding ENA-78/CXCL5 polypeptides may be accessed from public databases, e.g. Genbank, journal publications, etc. While various mammalian encoding ENA-78/CXCL5 polypeptides are of interest, for the treatment of human disease, generally the human protein will be used. Human ENA-78/CXCL5 genomic sequence may be found in Genbank, accession numbers L37036, U12709. Human ENA-78/CXCL5 coding sequence may be found in the Swiss-Prot Protein Knowledge base, accession number P42830.

ENA-78/CXCL5 polypeptide in its mature form consists of 78 amino acids and has a molecular weight of 8353 Da See, generally, Chang, et al., 1994, J. Biol. Chem. 269: 25277; and Walz, et al., 1991, J. Exp. Med. 174: 1355.

It is a feature of the invention that administration of IFN-γ has been discovered to markedly down-regulate expression of ENA-78/CXCL5 in patients with IPF. Thus, ENA-78/CXCL5 can be useful as an IFN-γ regulated biomarker for use in methods of the invention directed to determining a patient's response to IFN-γ therapy. Furthermore, antagonists of ENA-78/CXCL5 are useful in methods of the invention to treat patients suffering from IPF. Antagonists to ENA-78/CXCL5 are known and include anti-ENA-78/CXCL5 antibodies as well as CXCL8(3-73)K11R/G31P (Li, et al., 2002, Vet. Immunol. Immunopathol. 90(1-2): 65).

In addition to the CXC family of chemokines, additional agents have properties that can make them useful as IFN-γ regulated biomarkers for use in methods of the invention directed to determining a patient's response to IFN-γ therapy. Examples of such biomarkers are briefly described below.

IL-4

The nucleic acid sequences encoding IL-4 polypeptides may be accessed from public databases, e.g. Genbank, journal publications, etc. While various mammalian encoding ENA-78/CXCL5 polypeptides are of interest, for the treatment of human disease, generally the human protein will be used. Human IL-4 genomic sequence may be found in Genbank, accession number M13982. The corresponding coding sequence maybe found in Genbank, accession number 1310839.

IL-4 is a type II cytokine manufactured by activated T cells, mast cells, and basophils. See, generally, Brown, et al., 1997, Crit. Rev. Immunol. 17: 1; and Tepper, 1994, Res. Immunol. 144: 633. Although it induces a wide variety of biological responses, among its most important activities are its regulation of helper T cell differentiation to the TH2 type, and its regulation of the production of IgE and IgG1 by B cells.

It is a feature of the invention that a subset of patients with IPF demonstrate a reduction in IL-4 expression in response to IFN-γ therapy. Therefore, IL-4 can be useful as an

IFN-γ regulated biomarker in methods of the invention directed to determining a patient's response to IFN-γ therapy.

PDGF-B

The nucleic acid sequences encoding PDGF-B polypeptides may be accessed from public databases, e.g. Genbank, journal publications, etc. While various mammalian encoding PDGFB polypeptides are of interest, for the treatment of human disease, generally the human protein will be used. PDGFB coding sequence may be found in Genbank, accession number CAV02635. PDGFB genomic sequence may be found in Genbank, accession number Z81010.

PDGF (platelet-derived growth factor) comprises an entire family of homo- and heterodimers of two homologous genes, PDGF A chain and PDGF B chain, along with homodimers of PDGF-C. PDGF is an important regulator of connective tissue cells in embryogenesis, and is involved in the pathogenesis of a number of disease states. See, generally, Antoniades, 1983, Fed. Proc. 42: 2630; Betsholtz et al., 1997, Kidney Int. 51: 1361. PDGF and its receptors are elevated in various inflammatory disorders. PDGFB expression has been found to be elevated in the alveolar macrophages of individuals with idiopathic pulmonary fibrosis. See Nagaoka et al., 1990, J. Clin. Invest. 85: 2023. PDGF is believed to play an important role in the development of pulmonary fibrosis. PDGF has been found in bronchoalveolar lavage fluid in animal models of bleomycin-induced pulmonary fibrosis. (Maeda et al., 1996, Chest 109:780). Over-expression of PDGF-BB in rat lung has been shown to lead to pulmonary fibrosis. (Yoshida et al., 1995, Proc. Natl. Acad. Sci. USA 92(21):9570)

PDGF-B is useful as an IFN-γ regulated biomarker for use in methods of the invention directed to determining a patient's response to IFN-γ therapy, with down-regulation of PDGF-B expression in correlated with patient response to IN-γ therapy. Furthermore, antagonists of PDGF-B can be useful in methods of the invention to treat patients suffering from IPF. Antagonists to PDGF-B, including oligonucleotide and antibody antagonists, are also known. See, for example, Ostendorf, et al., 2001, J. Am. Nephrol. 12: 909, disclosing nuclease resistant aptamer; and Sjoblom, et al., 2001, Cancer Research 61: 5778, disclosing a low molecular weight inhibitor, ST1571.

Direct Biomarkers of Fibrotic Disorders

Procollagen III and elastin are both known to serve as direct markers of pulmonary fibrosis. Levels of procollagen III expression have been shown to correlate with an imbalance of ELR and non-ELR CXC chemokines in BALF in patients with suffering from acute respiratory distress syndrome. Keane et al., 2002, J. Immunol. 169(11):6515. Elastin is a chief component of lung interstitium, and is central to the morphology and function of the lung. Marked upregulation of elastin gene expression has been found to correlate with the histopathology of fibrotic lung disease. Hoff et al., 1999 Connect. Tissue Res. 40(2):145.

The roles of procollagen III and elastin as direct markers of pulmonary fibrosis can make them useful as IFN-γ regulated biomarkers for use in methods of the invention directed to determining a patient's response to IFN-γ therapy.

Pirfenidone and Analogs Thereof

Pirfenidone (5-methyl-1-phenyl-2-(1H)-pyridone) and specific pirfenidone analogs are useful for coadministration with agents of the invention for the treatment of fibrotic conditions, and have the following structure. 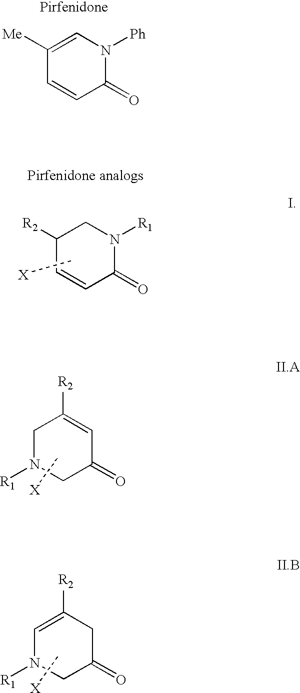
Descriptions for Substituents R₁, R₂, X
R₁: carbocyclic (saturated and unsaturated), heterocyclic (saturated or unsaturated), alkyls (saturated and unsaturated). Examples include phenyl, benzyl, pyrimidyl, naphthyl indolyl, pyrrolyl, furyl, thienyl, imidazolyl, cyclohexyl, piperidyl, pyrrolidyl, morpholinyl, cyclohexenyl, butadienyl, and the like.
R₁ can further include substitutions on the carbocyclic or heterocyclic moieties with substituents such as halogen, nitro, amino, hydroxyl, alkoxy, carboxyl, cyano, thio, alkyl, aryl, heteroalkyl, heteroaryl and combinations thereof, for example, 4-nitrophenyl, 3-chlorophenyl, 2,5-dinitrophenyl, 4-methoxyphenyl, 5-methylpyrrolyl, 2,5-dichlorocyclohexyl, guanidinyl-cyclohexenyl and the like.
R₂: alkyl, carbocylic, aryl, heterocyclic. Examples include: methyl, ethyl, propyl, isopropyl, phenyl, 4-nitrophenyl, thienyl and the like.
X: may be any number (from 1 to 3) of substituents on the carbocyclic or heterocyclic ring. The substituents can be the same or different. Substituents can include hydrogen, alkyl, heteroalkyl, aryl, heteroaryl, halo, nitro, carboxyl, hydroxyl, cyano, amino, thio, alkylamino, haloaryl and the like.
The substituents may be optionally further substituted with 1-3 substituents from the group consisting of alkyl, aryl, nitro, alkoxy, hydroxyl and halo groups. Examples include: methyl, 2,3-dimethyl, phenyl, p-tolyl, 4-chlorophenyl, 4-nitrophenyl, 2,5-dichlorophenyl, furyl, thienyl and the like.

Specific Examples include:

TABLE 1

| IA | IIB |
|---|---|
| 5-Methyl-1-(2′-pyridyl)-2- | 6-Methyl-1-phenyl-3- |
| (1H)pyridine, | (1H)pyridone, |
| 6-Methyl-1-phenyl-2-(1H)pyridone, | 5-Methyl-1-p-tolyl-3- |
| 5-Methyl-3-phenyl-1-(2′- | (1H)pyridone, |
| thienyl)-2-(1H)pyridone, | 5-Methyl-1-(2′-naphthyl)- |
| 5-Methyl-1-(2′-naphthyl)-2- | 3-(1H)pyridone, |
| (1H)pyridone, | 5-Methyl-1-phenyl-3- |
| 5-Methyl-1-p-tolyl-2-(1H)pyridone, | (1H)pyridone, |
| 5-Methyl-1-(1′naphthyl)-2- | 5-Methyl-1-(5′-quinolyl)- |
| (1H)pyridone, | 3-(1H)pyridone, |
| 5-Ethyl-1-phenyl-2-(1H)pyridone, | 5-Ethyl-1-phenyl-3- |
| 5-Methyl-1-(5′-quinolyl)-2- | (1H)pyridone, |
| (1H)pyridone, | 5-Methyl-1-(4′- |
| 5-Methyl-1-(4′-quinolyl)-2- | methoxyphenyl)-3-(1H)pyridone, |
| (1H)pyridone, | 4-Methyl-1-phenyl-3- |
| 5-Methyl-1-(4′-pyridyl)-2- | (1H)pyridone, |
| (1H)pyridone, | 5-Methyl-1-(3′-pyridyl)-3- |
| 3-Methyl-1-phenyl-2-(1H)pyridone, | (1H)pyridone, |
| 5-Methyl-1-(4′-methoxyphenyl)- | 5-Methyl-1-(2′-Thienyl)-3- |
| 2-(1H)pyridone, | (1H)pyridone, |
| 1-Phenyl-2-(1H)pyridone, | 5-Methyl-1-(2′-pyridyl)-3- |
| 1,3-Diphenyl-2-(1H)pyridone, | (1H)pyridone, |
| 1,3-Diphenyl-5-methyl-2- | 5-Methyl-1-(2′-quinolyl)-3- |
| (1H)pyridone, | (1H)pyridone, |
| 5-Methyl-1-(3′- | 1-Phenyl-3-(1H)pyridine, |
| trifluoromethylphenyl)-2- | 1-(2′-Furyl)-5-methyl-3- |
| (1H)-pyridone, | (1H)pyridone, |
| 3-Ethyl-1-phenyl-2-(1H)pyridone, | 1-(4′-Chlorophenyl)-5-methyl- |
| 5-Methyl-1-(3′-pyridyl)-2- | 3-(1H)pyridine. |
| (1H)pyridone, | |
| 5-Methyl-1-(3-nitrophenyl)-2- | |
| (1H)pyridone, | |
| 3-(4′-Chlorophenyl)-5-Methyl- | |
| 1-phenyl-2-(1H)pyridone, | |
| 5-Methyl-1-(2′-Thienyl)-2- | |
| (1H)pyridone, | |
| 5-Methyl-1-(2′-thiazolyl)-2- | |
| (1H)pyridone, | |
| 3,6-Dimethyl-1-phenyl-2- | |
| (1H)pyridone, | |
| 1-(4′Chlorophenyl)-5-Methyl- | |
| 2-(1H)pyridone, | |
| 1-(2′-Imidazolyl)-5-Methyl- | |
| 2-(1H)pyridone, | |
| 1-(4′-Nitrophenyl)-2- | |
| (1H)pyridone, | |
| 1-(2′-Furyl)-5-Methyl-2- | |
| (1H)pyridone, | |
| 1-Phenyl-3-(4′-chlorophenyl)- | |
| 2-(1H)pyridine. | |

U.S. Pat. Nos. 3,974,281; 3,839,346; 4,042,699; 4,052,509; 5,310,562; 5,518,729; 5,716,632; and 6,090,822 describe methods for the synthesis and formulation of pirfenidone and specific pirfenidone analogs in pharmaceutical compositions suitable for use in the methods of the present invention.

Variants

The polypeptides of the present invention may be any of the polypeptides as they naturally occur, in their recombinant forms, and the derivatives thereof so far as they have substantially similar biological activity, particularly human biological activity.

In particular, the IFN-γ to be used in the compositions of the present invention may be any of natural IFN-γs, recombinant IFN-γs and the derivatives thereof so far as they have a IFN-γ activity, particularly human IFN-γ activity. Human IFN-γ exhibits the antiviral and anti-proliferative properties characteristic of the interferons, as well as a number of other immunomodulatory activities, as is known in the art. Although IFN-γ is based on the sequences as provided above, the production of the protein and proteolytic processing can result in processing variants thereof. The unprocessed sequence provided by Gray et al., supra. consists of 166 amino acids (aa). Although the recombinant IFN-γ produced in E. coli was originally believed to be 146 amino acids, (commencing at amino acid 20) it was subsequently found that native human IFN-γ is cleaved after residue 23, to produce a 143 aa protein, or 144 aa if the terminal methionine is present, as required for expression in bacteria. During purification, the mature protein can additionally be cleaved at the C terminus after reside 162 (referring to the Gray et al. sequence), resulting in a protein of 139 amino acids, or 140 amino acids if the initial methionine is present, e.g. if required for bacterial expression. The N-terminal methionine is an artifact encoded by the mRNA translational “start” signal AUG which, in the particular case of E. coli expression is not processed away. In other microbial systems or eukaryotic expression systems, methionine may be removed.

For use in the subject methods, any of the native IFN-γ peptides, modifications and variants thereof, or a combination of one or more peptides may be used. IFN-γ peptides of interest include fragments, and can be variously truncated at the carboxy terminal end relative to the full sequence. Such fragments continue to exhibit the characteristic properties of human gamma interferon, so long as amino acids 24 to about 149 (numbering from the residues of the unprocessed polypeptide) are present. Extraneous sequences can be substituted for the amino acid sequence following amino acid 155 without loss of activity. See, for example, U.S. Pat. No. 5,690,925, herein incorporated by reference. Native IFN-γ moieties include molecules variously extending from amino acid residues 24-150; 24-151, 24-152; 24-153, 24-155; and 24-157. Any of these variants, and other variants known in the art and having IFN-γ activity, may be used in the present methods.

The sequence of the polypeptides may be altered in various ways known in the art to generate targeted changes in sequence. A variant polypeptide will usually be substantially similar to the sequences provided herein, i.e. will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. In particular, the invention contemplates in one embodiment the use of IFN-γ variants with one or more non-naturally occurring glycosylation and/or pegylation sites that are engineered to provide glycosyl- and/or PEG-derivatized polypeptides with reduced serum clearance, such as the IFN-γ polypeptide variants described in International Patent Publication No. WO 01/36001.

Included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g. D-amino acids or non-naturally occurring synthetic amino acids. The protein may be pegylated to enhance stability.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Method of Treating Idiopathic Pulmonary Fibrosis

The present invention provides methods of treating idiopathic pulmonary fibrosis (IPF). The methods generally involve administering an effective amount of one or more of IFNγ, I-TAC/CXCL11, antagonists of ENA-78/CXCL5, antagonists of IL-4, and an antagonist of PDGF-B to an individual having IPF.

A diagnosis of IPF may be confirmed by the finding of usual interstitial pneumonia (UIP) on histopathological evaluation of lung tissue obtained by surgical biopsy. The criteria for a diagnosis of IPF are known. Ryu et al. (1998) Mayo Clin. Proc. 73:1085-1101.

Alternatively, a diagnosis of IPF is a definite or probable IPF made by high resolution computer tomography (HRCT). In a diagnosis by HRCT, the presence of the following characteristics is noted: (1) presence of reticular abnormality and/or traction bronchiectasis with basal and peripheral predominance; (2) presence of honeycombing with basal and peripheral predominance; and (3) absence of atypical features such as micronodules, peribronchovascular nodules, consolidation, isolated (non-honeycomb) cysts, ground glass attenuation (or, if present, is less extensive than reticular opacity), and mediastinal adenopathy (or, if present, is not extensive enough to be visible on chest x-ray). A diagnosis of definite IPF is made if characteristics (1), (2), and (3) are met. A diagnosis of probable IPF is made if characteristics (1) and (3) are met.

The antagonist of ENA-78/CXCL5, IL-4, or PGDF-B can be an antibody or fragment thereof, to ENA-78/CXCL5, IL-4, or PGDF-B, respectively.

In some embodiments, pirfenidone or a pirfenidone analog is co-administered for the duration of treatment with IFN-γ, I-TAC/CXCL11, an antagonist of ENA-78/CXCL5, an antagonist of IL-4, an antagonist of PDGF-B, or a combination thereof.

In all embodiments of the invention where a combination of agents are administered, the agents may be administered in any suitable manner. For example, administering some or all of the agents separately so that the agents are combined in situ is within the scope of the invention. Alternatively, some or all of the agents may be combined as an admixture before administration to the patient.

IFN-γ, I-TAC/CXCL11, antagonists of ENA-78/CXCL5, antagonists of IL-4, antagonists of PDGF-B, and combinations thereof are administered in effective amounts. In some embodiments, an effective amount is an amount effective to increase the probability of survival of an individual having IPF by at least about 10%, at least about 15%, at least about 20%, or at least about 25%, or more, compared to the expected probability of survival without administration of IFN-γ, I-TAC/CXCL11, antagonists of ENA-78/CXCL5, antagonists of IL-4, and combinations thereof. Thus, the increased probability of survival of an individual having IPF and administered with an effective amount of IFN-γ, I-TAC/CXCL11, an antagonist of ENA-78/CXCL5, an antagonist of IL-4, an antagonist of PDGF-B, or combinations thereof is at least about 10%, at least about 15%, at least about 20%, or at least about 25%, or more, compared to the expected probability of survival without administration of IFN-γ.

In some embodiments, an effective amount of IFN-γ, I-TAC/CXCL11, an antagonist of ENA-78/CXCL5, an antagonist of IL-4, an antagonist of PDGF-B, or a combination of the foregoing agents is an amount that reduces the risk of death in an individual with IPF. The risk of death in an individual having IPF and treated with one of the foregoing agents or a combination thereof is reduced at least 2-fold, at least 2.5-fold, at least 3-fold, at least 3.5-fold, or at least 4-fold, or less, compared to the expected risk of death in an individual having IPF and not treated with one of the foregoing agents or a combination thereof.

In some embodiments, an effective amount of IFN-γ, I-TAC/CXCL11, an antagonist of ENA-78/CXCL5, an antagonist of IL-4, an antagonist of PDGF-B, or a combination of the foregoing agents is an amount that reduces or avoids dysregulated angiogenesis in the pulmonary vasculature of the patient.

In some embodiments, the effective amounts of a combination of administered agents are synergistically effective to reduce or avoid dysregulated angiogenesis in the pulmonary vasculature of the patient.

In some embodiments, an effective amount of IFN-γ, I-TAC/CXCL11, an antagonist of ENA-78/CXCL5, an antagonist of IL-4, an antagonist of PDGF-B, or a combination of the foregoing agents is an amount that reduces the risk of morbidity due to infection in the patient.

The administration of the agents of the invention to patients may continue for any length of treatment. In some embodiments, treatment with the agents of the invention is maintained for the entirety of the remaining life of the patient.

Methods of Diagnosing Patients' Responses to IFN-γ Therapy

The invention further provides methods for evaluating patient response to treatment with IFN-γ by analyzing expression of I-TAC/CXCL11, ENA-78/CXCL5, or both, in an IFN-γ-treated patient. Increased I-TAC expression, decreased ENA-78 expression, or both, as compared to a control expression, is correlated with patient response to IFN-γ treatment.

Post-treatment expression levels of I-TAC/CXCL11, ENA-78/CXCL5, or both, are compared with a control expression, for example, a baseline expression obtained from the patient prior to commencement of IFN-γ treatment. The results of the correlation can be used to develop strategies to increase, decrease, or leave unchanged the IFN-γ dosage that is administered to the patient. Comparison can be made between baseline, pre-treatment expression, and post-treatment expression of I-TAC/CXCL11 and/or ENA-78/CXCL5 determined from about 2 hours to about 4 weeks after a patient has begun treatment with interferon gamma. In another embodiment, two or more post-treatment samples can be analyzed and compared to monitor patient response to continued IFN-γ therapy. The results of the correlation can be used to develop strategies to discontinue therapy, modify dose, and the like.

The levels of I-TAC/CXCL11 and/or ENA-78/CXCL5 expression can be determined from any suitable source such as exhaled breath condensate, bronchoalveolar lavage fluid or pelleted cells, transbronchial biopsy tissue, or blood sample, including serum obtained from the patient. Relative expression of mRNA can be analyzed in tissue or cellular samples and/or protein can be analyzed in breath condensates, lavage fluids or blood components such as serum or plasma. In some embodiments, expression is analyzed by measuring protein in breath condensate.

Where mRNA is determined, a preferred method is TaqMan Real Time PCR, due to it's greater sensitivity over other PCR methods.

The invention further provides methods for evaluating IPF patient response to IFN-γ therapy by comparing post-treatment levels of IL-4 and/or PDGF-B with pretreatment-levels, and correlating a relative decrease in IL-4 and/or decrease in PDGF-B with patient response to IFN-γ.

The invention further provides methods for evaluating patient response to treatment with IFN-γ by analyzing expression of elastin, procollagen III, or both, in an IFN-γ-treated patient. The methods further provide correlating decreased elastin expression, decreased procollagen III expression, or both, as compared to a control expression, with patient response to IFN-γ treatment.

Dosages, Formulations, and Routes of Administration:

IFN-γ is administered to individuals in a formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

In the subject methods, the active agent(s) may be administered to the host using any convenient means capable of resulting in the desired therapeutic effect. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the agents can be achieved in various ways, including oral, buccal, rectal, parenteral, intraperitoneal, intradermal, transdermal, intracheal, etc., adlministration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Effective dosages of IFN-γ can range from about 0.5 μg/m² to about 500 μg/m², usually from about 1.5 μg/m² to 200 μg/m², depending on the size of the patient. This activity is based on 10⁶ international units (IU) per 50 μg of protein. Additional agents such as I-TAC are administered in the rang of from 0.005 μg/m² to about 50,000 μg/m², preferable from about 0.05 μg/m² to about 5,000 μg/m².

Those of skill will readily appreciate that dose levels can vary as a function of the specific compound, the severity of the symptoms and the susceptibility of the subject to side effects. Preferred dosages for a given compound are readily determinable by those of skill in the art by a variety of means. A preferred means is to measure the physiological potency of a given compound.

In specific embodiments of interest, IFN-γ is administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments of interest, IFN-γ1b is administered.

Where the dosage is 200 μg IFN-γ per dose, the amount of IFN-γ per body weight (assuming a range of body weights of from about 45 kg to about 135 kg) is in the range of from about 4.4 μg IFN-γ per kg body weight to about 1.48 μg IFN-γ per kg body weight.

The body surface area of subject individuals generally ranges from about 1.33 m² to about 2.50 m². Thus, dosage groups (based on administration of 200 μg IFN-γ per dose) range from about 150 μg/m² to about 80 μg/m². For example, dosage groups range from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μg/m² to about 150 μg/m².

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide (e.g., a polynucleotide encoding IFN-γ), it may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the therapeutic DNA, then bombarded into skin cells. Of particular interest in these embodiments is use of a liver-specific promoter to drive transcription of an operably linked IFN-γ coding sequence preferentially in liver cells.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

In particular embodiments of interest, IFN-γ is administered as a solution suitable for subcutaneous injection. For example, IFN-γ is in a formulation containing 40 mg mannitol/mL, 0.72 mg sodium succinate/mL, 0.10 mg polysorbate 20/mL. In particular embodiments of interest, IFN-γ is administered in single-dose forms of 200 μg/dose subcutaneously.

Multiple doses of IFN-γ can be administered, e.g., IFN-γ can be administered once per month, twice per month, three times per month, once per week, twice per week, three times per week, four times per week, five times per week, six times per week, or daily, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In particular embodiments of interest, IFNγ is administered three times per week over a period of at least about 1 year.

Additional Agents

IFN-γ may be co-administered with one or more additional agents in the treatment of IPF. Suitable additional agents include corticosteroids, such as prednisone. When co-administered with IFN-γ, prednisone is administered in an amount of 7.5 mg or 15 mg daily, administered orally.

Subjects Suitable for Treatment

The methods of the invention are suitable for treatment and analysis of individuals diagnosed as having IPF. The methods are also suitable for treatment of individuals having IPF who were previously treated with corticosteroids within the previous 24 months, and who failed to respond to previous treatment with corticosteroids. Subjects that are particularly amenable to treatment with a method are those that have at least 55% of the predicted FVC. Also suitable for treatment are subject that have at least 60% of the predicted FVC, or from 55% to 70% of the predicted FVC. The percent predicted FVC values are based on normal values, which are known in the art. See, e.g., Crapo et al. (1981) Am. Rev. Respir. Dis. 123:659-664. FVC is measured using standard methods of spirometry.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Treatment of IPF with IFN-γ

### Materials and Methods

Study Population

Male and female patients were those ages 20-79 with idiopathic pulmonary fibrosis. Patients aged 20-34 were diagnosed by open or video-assisted thoracoscopic (VATS) lung biopsy or by transbronchial biopsy to be eligible. Diagnosis was made by high resolution computer tomographic scan showing definite or probable IPF and either open or VATS lung biopsy showing definite or probable usual interstitial pneumonia (UIP) within 30 months prior to screening; or non-diagnostic transbronchial biopsy to exclude other conditions within 30 months prior to screening and abnormal PFTs (reduced FVC or decreased DL_{co} or impaired gas exchange with rest or exercise) and 2 of the following: age greater than 50 years, insidious onset of otherwise unexplained dyspnea on exertion, and bibasilar, inspiratory crackles (dry or “Velcro” type in quality). Patients had clinical symptoms consistent with IPF of ≧3 months duration and had worsening disease within the past year.

Patients included in the study failed to show improvement after an adequate course of steroids that was completed within the 24 months prior to treatment in the present protocol. Failure to show “improvement” refers to failure to show an increase of ≧10% in the percent predicted FVC from baseline value (before steroids started) to any point after the steroid administration period and before randomization. Patients who showed ≧10% improvement, then returned to the baseline value, despite the continuation of the same dose of steroids that was associated with improvement, were eligible. For patients with a diagnosis of IPF established within the past year, an “adequate course” of steroids is a total oral dose of 1800 mg of prednisone or its equivalent administered over a period of no less than 1 month and no greater than 3 months. For patients with a diagnosis of IPF established more than 1 year prior to treatment, an “adequate course” of steroids is a total oral dose of 1800 mg of prednisone or its equivalent administered within a 6 month period.

Exclusion Criteria

Patients with any of the following were excluded from the study:
(1) History of clinically significant environmental exposure known to cause pulmonary fibrosis (drugs, asbestos, beryllium, radiation, domestic birds, etc.);
(2) Known explanation for interstitial lung disease, other than IPF, including but not limited to radiation, sarcoidosis, hypersensitivity pneumonitis, bronchiolitis obliterans organizing pneumonia (BOOP), and cancer;
(3) Diagnosis of any connective tissue disease (scleroderma, systemic lupus erythematosus, rheumatoid arthritis, etc.) according to American College of Rheumatology criteria;
(4) Forced expiratory volume in the first second (FEV1)/forced vital capacity (FVC) ratio<0.6 at Screening (post-bronchodilator);
(5) Patients with a residual volume>120% of predicted at Screening (pre-bronchodilator);
(6) Evidence of active infection, including bronchitis, sinusitis, urinary tract infection (UTI), and cellulitis within 1 week prior to treatment;
(7) Any condition other than IPF which, in the opinion of the site Principal Investigator (PI), is likely to result in the death of the patient within the next year;
(8) History of unstable or deteriorating cardiac or neurologic disease, including but not limited to:
   a) Myocardial infarction, coronary artery bypass surgery, or angioplasty within the past 6 months;
   b) Congestive heart failure requiring hospitalization within the past 6 months;
   c) Uncontrolled arrhythmias;
   d) Transient ischemic attacks (TIAs);
(9) Any cardiac or neurologic condition which, in the opinion of the site PI, might be significantly exacerbated by the known flu-like syndrome associated with the administration of IFN-γ 1b;
(10) History of peripheral vascular disease which, in the opinion of the site PI, might be exacerbated by the known flu-like syndrome associated with the administration of IFN-γ1b;
(11) History of CNS disorder which, in the opinion of the site PI, might be exacerbated by the known flu-like syndrome associated with the administration of IFN-γ1b. In addition, patients with the following conditions should be excluded:
   a) History of multiple sclerosis;
   b) Seizures within the past 10 years or taking anti-seizure medication;
(12) History of severe or poorly controlled diabetes;
(13) Pregnancy or lactation. Females of childbearing potential were required to have a negative serum or urine pregnancy test prior to treatment and must agree to practice abstinence or prevent pregnancy by at least a barrier method of birth control for the duration of the study;
(14) Any of the following liver function test criteria above specified limits: Total bilirubin≧1.5×ULN; aspartate or alanine aminotransferases (AST, SGOT or ALT, SGPT)>3×ULN; alkaline phosphatase>3×ULN; and albumin<3.0 mg/dL at Screening;
(15) Hematology outside of specified limits: WBC<2,500/mm3, hematocrit<30% or >59%, platelets<100,000/mm3 at Screening;
(16) Creatinine>1.5×ULN at Screening;
(17) Prior treatment with IFN-γ1b, beta interferon (Avonex), or other interferons;
(18) Investigational therapy for any indication within 28 days prior to treatment;
(19) Use of azathioprine, colchicine, cyclophosphamide, cyclosporine, D-penicillamine, methotrexate, or N-acetyl cysteine within 6 weeks prior to treatment;
(20) Investigational therapy, including pirfenidone, within 6 months prior to treatment;
(21) Patients who, in the opinion of the site PI, are not suitable candidates for enrollment or would not comply with the requirements of the study.
   Primary Endpoints

The primary endpoints of the study included progression-free survival time (e.g., time from baseline to death or disease progression). Disease progression was defined as the occurrence of either of the following: a decrease in % predicted FVC of 10% or more compared to baseline on two consecutive occasions 4-14 weeks apart; an increase in A-a gradient of 5 mm Hg or more compared to baseline on two separate occasions 4-14 weeks apart.

Secondary Endpoints

Secondary endpoints included the following:
(1) Transitional dyspnea index (DI) at Week 48;
(2) Progression-free survival time with disease progression defined by the presence of any two of the following:
   (a) Decrease of 10% or more in percent predicted FVC;
   (b) Increase of 5 mmHg or more in A-a gradient;
   (c) Decrease of 15% or more in single breath DL_{CO};
(3) Change from baseline to Week 48 in DL_{CO} (numerical value);
(4) Change from baseline to Week 48 in FVC (numerical value);
(5) Change from baseline to Week 48 in A-a gradient (numerical value);
(6) Quality of Life as assessed by the St. George's Respiratory Questionnaire total score change from baseline to Week 48;
(7) Survival time from randomization through clinical data cutoff, summarized by treatment group;
(8) Response status of lung fibrosis as assessed by HRCT (better, same, worse) at 48 weeks compared to baseline;
(9) Most severe requirement for use of outpatient oxygen (none, with activity, at rest) during each month on study, compared between treatment groups.
   Safety Observations

Patients were evaluated at Weeks 1 and 2, and at monthly visits thereafter to assess adverse events. Laboratory tests, including a complete blood count; routine chemistry tests including creatinine; liver function tests; cholesterol; triglycerides; and urinalysis were measured at baseline, Weeks 1, 2, 4, 12, and every 12 weeks thereafter. Thyroid function tests were performed at baseline, Week 12 Month 3), Week 24 (Month 6), Week 48 and every 6 months thereafter. Any Serious Adverse Events and Grade IV toxicities were reported in real time to the Sponsor or its designee regardless of relationship to study drug.

Efficacy Observations

Patients were subjected to pulmonary function tests (spirometry, DL_{CO}) and resting arterial blood gases assessed at baseline and every 12 weeks (3 months) thereafter. Dyspnea (modified MRC scale) was assessed at baseline and every 4 weeks (monthly) thereafter. Dyspnea (BDI/TDI and the UCSD Shortness of Breath Questionnaire) was assessed at baseline and every 12 weeks (3 months) thereafter. Quality-of-life questionnaires (SF-36 and SGRQ) were given at baseline and every 12 weeks (3 months) thereafter. Oxygen use was monitored daily. HRCT scans were performed at baseline (prior to initiation of treatment) and at 48 weeks.

Study Design

A randomized, double-blind, placebo-controlled study of 330 patients with randomization balanced by study site and for smoking status. Patients were assigned to one of two groups: Group 1: 200 μg IFN-γ1b subcutaneous administration three times a week; Group 2: placebo, subcutaneous administration of saline three times a week (tiw).

The study comprised three periods: the Screening Period (up to 28 days duration), the Study Period (up to 37 months duration), and the Long-Term Follow-Up Period (5 years). During the Study Period, patients were dosed with study drug tiw for up to 3 years. The final analysis was conducted when the 306^{th} patient had been followed for 48 weeks and included data from all patients randomized. Patients who withdrew from study treatment early had a complete post-treatment evaluation visit 12 weeks (3 months) after their last treatment and then visited every 12 weeks (3 months) thereafter for assessment of primary and secondary endpoints as well as medications used to treat IPF.

Study treatment continued until the Study Completion Visit, and the Study Period ended with the Follow-Up Visit conducted 28 days following the Study Completion Visit. Subsequent to the Study Period, patient vital status will be assessed every 6 months for 5 years during the Long-Term Follow-Up Period. A Data and Safety Monitoring Board (DSMB) monitored patient safety regularly.

Patients may be taldng up to 15 mg of prednisone per day at study entry and should remain on the same dose (entry level) of steroids throughout the study. Treatment with colchicine, cytotoxic drugs, cyclosporine, N-acetyl cysteine, or other experimental therapies will not be allowed.

Data Analysis

The primary efficacy endpoint is the time to first occurrence of disease progression or death, as assessed by the Cox proportional hazards model.

Results

No statistically significant difference was apparent in the progression-free survival times of the treatment and placebo groups. Nevertheless, a statistically significant improvement in probability of survival was apparent in certain subpopulations of the treatment and placebo groups.

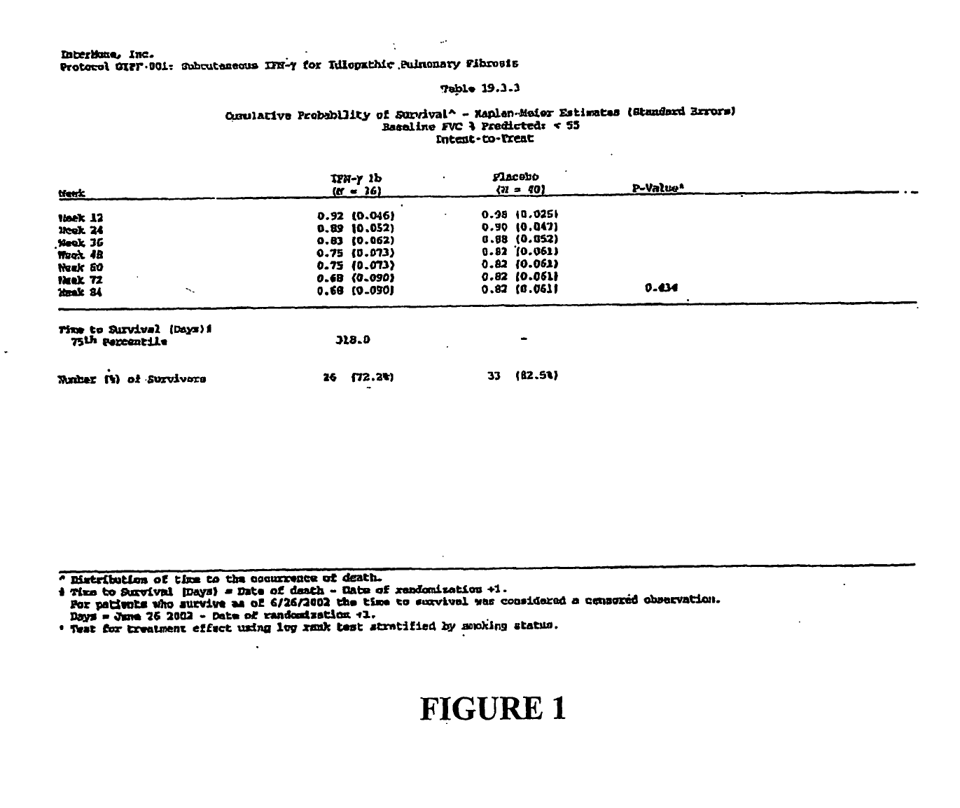

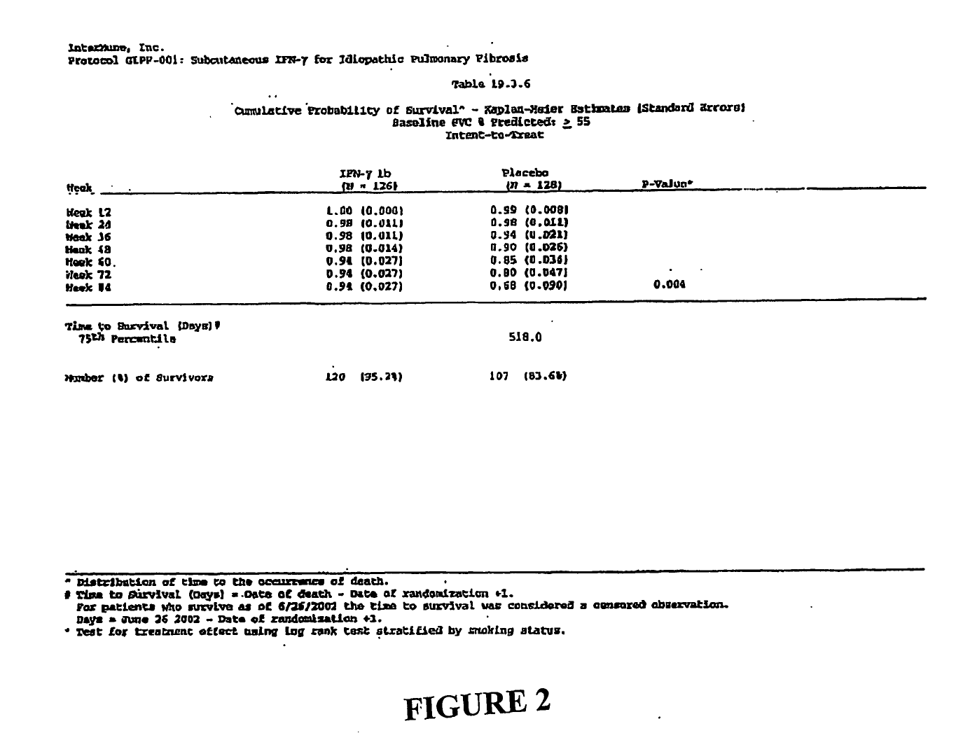

The results for patient survival are shown in FIGS. 1 and 2. FIG. 1 presents the data for individuals who had a % predicted FVC of less than 55 at the beginning of treatment. Individuals (N=36) treated with IFN-γ 1b and having a % predicted FVC of less than 55% had a probability of 72.2% survival, while placebo controls (N=40), had an 82.5% probability of survival (p=0.434). Thus, the observed risk of death among individuals with IPF and having an FVC of less than 55% of the predicted normal value was 27.8%, while the risk of death of the placebo controls was 17.5%. There is no statistical evidence that IFN-γ 1b has a survival effect in these patients.

### Example 2

### Analysis of Biomarkers in IPF patients treated with IFN-γ

Molecular, cellular, and whole animal studies have suggested multiple pathways may contribute to fibrosis related to the production and deposition of extracellular matrix (i.e., procollagens and elastin) in the lung. These molecular factors include growth factors (i.e., no TGF-β, CTGF, and PDGF) and cytokines/chemokines associated with inflammation, cellular trafficking, angiogenesis, and immunity. See, for example, Keane, et al., 2000, Inflammation, injury, and repair. In: J. F. Murray et al., editors. Textbook of Respiratory Medicine 3rd Edition. W. B. Saunder Co., Philadelphia, Pa. 495-538; and Keane, et al., 2003, Cytokine biology and the pathogenesis of interstitial lung diseas. In: M. I. Schwarz and T. E. King, editors. Interstitial Lung Disease, 4th ed. B. C. Decker, Inc., Hamilton, Ontario, Canada, 2003.

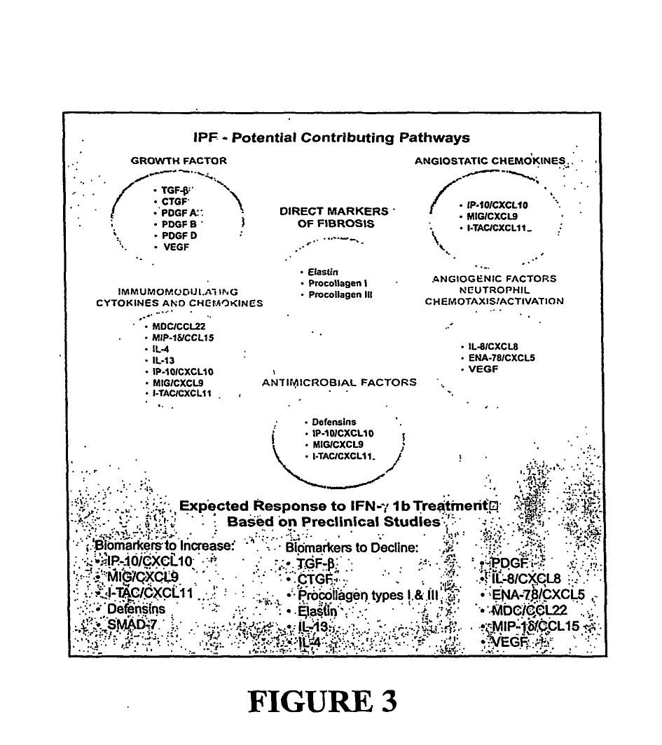

Interferon gamma-1b (IFN-γ1b) is a pleiotropic cytokine with antimicrobial, anti-fibrotic/antiproliferative, and immunomodulator properties. IFN-γ1b is approved by the FDA for the treatment of chronic granulomatous disease and malignant osteopetrosis. IFN-γ1b reduces the incidence and severity of infections in patients with chronic granulomatous disease and decreases the progression of malignant osteopetrosis. Preclinical studies have shown that IFN-γ affects a number of molecules associated with fibrosis, including down-regulation of procollagens, elastin, TGF-β, CTGF, PDGF, ENA-78/CXCL5, IL-8/CXCL8, MDC/CCL22, MIP-1δ/CCL15, Il-4, and Il-13; and up-regulation of defensins, SMAD-7, and interferon-inducible CXC chemokines MIG/CXCL9, IP-10/CXCL10, and I-TAC/CXCL1. (See FIG. 3).

Ziesche and associates treated 18 patients with IFF who had failed conventional immunosuppressive therapy with either variable-dose methylprednisolone (n=9) or low-dose methylprednisolone and IFN-γ1b (n=9). The patients who were treated with IFN-γ1b and analyzed using traditional PCR techniques, demonstrated a down-regulation of mRNA for TGF-β and CTGF from transbronchial biopsies at 6 months and improvement in FVC and resting and exercise oxygenation at 46 weeks. See, Ziesche et al., 1999, N Engl J Md 341(17):1264-9, “A preliminary study of long-term treatment with interferon gamma-1b and low-dose prednisolone in patients with idiopathic pulmonary fibrosis”.

To confirm the results of Ziesche et al. and to extend these studies to include other biomarkers associated with fibrosis, aberrant vascular remodeling, inflammation and antimicrobial activity, a multicenter, randomized, placebo-controlled study was conducted. The purpose of the study was to characterize biologic and clinical effects of IFN-γ 1b administered subcutaneously to patients with IPF.

Study Population

A randomized, double-blind, placebo-controlled study of 33 patients was conducted. Patients were assigned to one of two groups: Group 1: 200 μg IFN-γ1b subcutaneous administration three times a week; Group 2: placebo, subcutaneous administration of saline three times a week (tiw).

The study comprised three periods: the Screening Period (lip to 28 days duration), the Study Period (6 months duration), and an optional open-label extension for additional 6 months (ongoing). During the Study Period, patients were dosed with study drug three times a week for up to 6 months. Data from all patients randomized was analyzed.

Male and female patients, aged 20-79, with idiopathic pulmonary fibrosis were randomly assigned into control and placebo study groups. Patient eligibility criteria included definite or probable diagnosis of IPF, with IPF symptoms for at least three months, and worsening in the past year, failure to respond to corticosteroid therapy, definite or probable IPF as determined by high-resolution computed tomographic scan (HRCT); lung function parameters of FVC at baseline of 50% or more and 90% or less than predicted value, DL_{CO} 25% or more of the predicted value at screening, and PaO₂ of greater than 55 mm Hg at rest. Patients had were able and willing to take 10 mg prednisone daily for at least 21 days prior to bronchoscopy and to continue the same dose until the end of the six-month study. Patient disposition, treatment compliance, and characteristics are shown in the tables below.

Patients followed a schedule that included an initial screening visit; baseline visit to obtain samples for baseline analyses, bronchoscopy on day −7, treatment on day 1, visits at weeks 1, 2, 4, 8, 12, 16, and 20, 6-month visit at week 22-23 to collect clinical endpoints; bronchoscopy for biomarker endpoints at week 23-24, and either end treatment visit or continue into the optional open label phase of the study.

Patient Disposition
Patient population - 32 patients at 15 sites who received
at least one dose of study drug

| | IFN-γ | Placebo |
|---|---|---|
| Patient Status | 1b (n = 17) | (n = 15) |
| Randomized | 17 | 15 |
| Received study treatment | 17 | 15 |
| Death | 0 | 1 |
| Discontinued due to adverse event | 0 | 1 |
| Baseline bronchoscopy | 17 | 15 |
| Week 23/24 bronchoscopy | 17 | 13 |

Treatment Compliance

| Percent of Scheduled | IFN-γ 1b | Placebo |
|---|---|---|
| Doses Received | (n = 17) | (n = 15) |
| Mean | 98% | 98% |
| ≧80% | 17 (100%) | 15 (100%) |

Demographics and Baseline Characteristics

| | IFN-γ 1b | Placebo |
|---|---|---|
| Demographics | (n = 17) | (n = 15) |
| Age (yr, mean) | 64.1 | 63 |
| 51-60, no. (%) | 5 (29) | 7 (47) |
| 61-70, no. (%) | 7 (41) | 5 (33) |
| 71-80, no. (%) | 5 (29) | 3 (20) |
| Sex [no. (%)] | | |
| Male | 12 (71) | 8 (53) |
| Female | 5 (29) | 7 (47) |
| Race [no. (%)] | | |
| Caucasian | 16 (94) | 13 (87) |
| Asian | 1 (6) | 0 (0) |
| Hispanic or Latino | 0 (0) | 2 (13) |
| Patient Status | | |
| Time since Dx of IPF (d ± SD) | 340 ± 261 | 377 ± 396 |
| | 671 ± 447 | 866 ± 525 |

Diagnosis of IPF: Tissue Diagnosis and HRCT

| | IFN-γ 1b | Placebo |
|---|---|---|
| Procedure | (n = 17) | (n = 15) |
| Definite IPF by HRTC, no. (%) | | |
| Surgical lung biopsy only | 4 (24) | 6 (40) |
| Transbronchial biopsy only | 7 (41) | 6 (40) |
| Both | 1 (6) | 0 (0) |
| Probable IPF by HRTC, no. (%) | | |
| Surgical lung biopsy only | 3 (18) | 3 (20) |
| Transbronchial biopsy only | 1 (6) | 0 (0) |
| Both | 1 (6) | 0 (0) |

Primary Endpoints

The primary endpoints of this study included change from baseline in the level of mRNA transcription in lung tissue, represented by relative expression (RE) calculations for TGF-β and CTGF in patients who received at least 80% of the study doses.

Secondary Endpoints

Secondary endpoints of this study included change in biomarkers from baseline at six months in the relative expression (RE) of mRNA in lung tissue biopsy (TBBx) and in bronchoalveolar lavage cell pellet (BAL), or in protein levels in the lavage fluid (BALF) and plasma of the study patients. Specific biomarkers evaluated included:

| | | |
|---|---|---|
| Type I Procollagen | MIG/CXCL9 | MDC/CCL22 |
| Type III Procollagen | I-TAC/CXCL11 | MIP-1δ/CCL15 |
| Elastin | IL-8/CXCL8 | interferon gamma |
| TGF-β | ENA-78/CXCL5 | SMAD = 7 |
| CTGF | Defensins (bioassay) | VEGF |
| PDGF-A, PDGF-B, PDGF-D | IL-4 | |
| IP-10/CXCL10 | IL-13 | |

Additional Secondary Endpoints were measured by change from baseline at 6 months (except where noted):

Percent predicted FVC

Resting A-a gradient

Percent predicted DL_{CO}

Dyspnea (MRC, TDI, UCSD SOBQ)

Most severe oxygen use (none, with activity, at rest) at each month

Maximum oxygen flow rate at each month

Distance walked in 6-minute Walk Test (6MWT)

Laboratory Analysis:

mRNA was measured by real time quantitative PCR (TAQ MAN®) (Gene Link, Hawthorne, N.Y.) using the ABI Prism Analyzer (Applied Biosystems, Foster City, Calif.). This type of PCR analysis is more sensitive than traditional PCR for detecting mRNA. A housekeeping gene is used to standardize the assay, and the quantitation is an indicator of relative amounts of mRNA, not absolute amounts. The data are shown as relative expression: where 1 is equal to no change in expression, a value >1 indicates an increase in expression, and a value <1 indicates a decrease in expression.

Protein analysis was by ELISA for all protein molecules except defensins. Defensins were quantitiated using a bioassay.

Statistical Analysis:

Relative expression of mRNA was made as the change in mRNA transcription levels in lung tissue from baseline to six months post onset of therapy was compared between IFN-γ1b and placebo groups using the analysis of covariance (ANCOVA), with baseline transcription level as the covariate. If the p-value for the covariate was greater than 0.10, then the covariate was dropped from the statistical model. If the assumption of normality failed, then nonparametric methods were used. For all mRNA data analyses, samples with an inadequate quantity of the housekeeping gene were excluded from the analyses. All mRNA transcription data (primary and secondary) were assessed on both continuous and categorical scales.

Other Variables:

Continuous measures were compared using ANCOVA, with the same assumptions for the covariate and normality as described above for mRNA data. Treatment groups were compared with respect to categorical values using Fisher's Exact exact test for binary outcomes and the Wilcoxon Rank-Sum test for ordered categorical outcomes. For each of the continuous and categorical outcome variables, final or “endpoint” evaluations were used in the analysis to incorporate data from dropouts. Values were carried forward from the date of last visit.

Results:

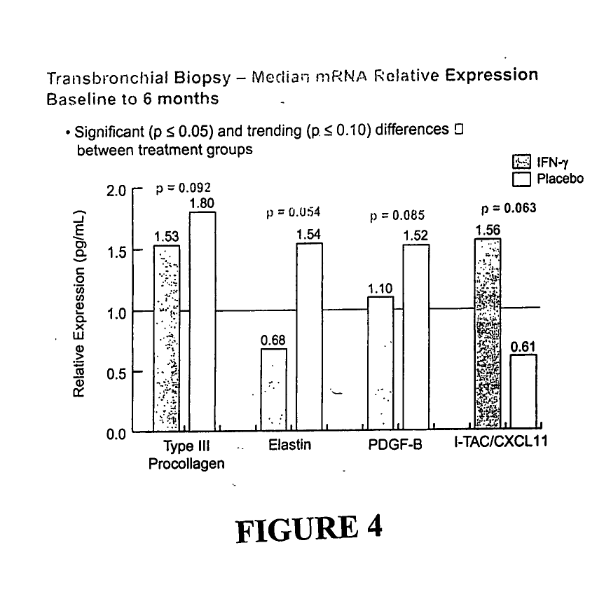

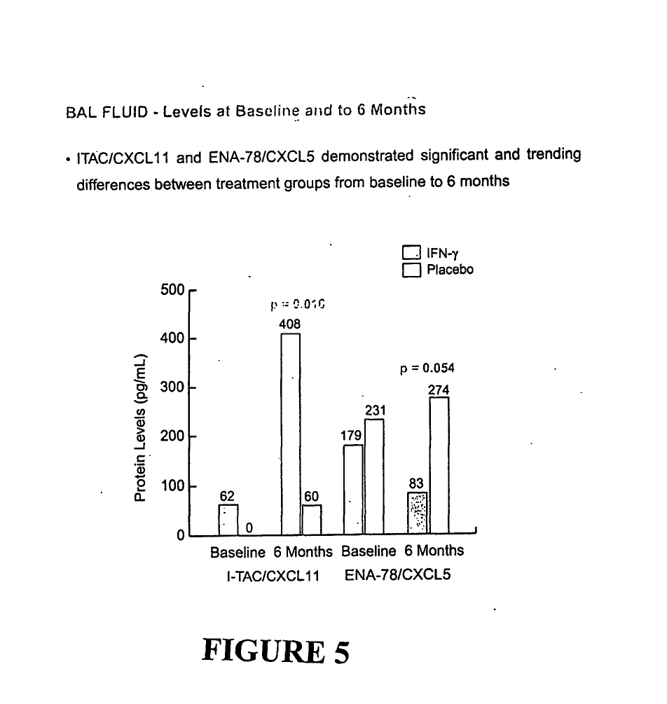

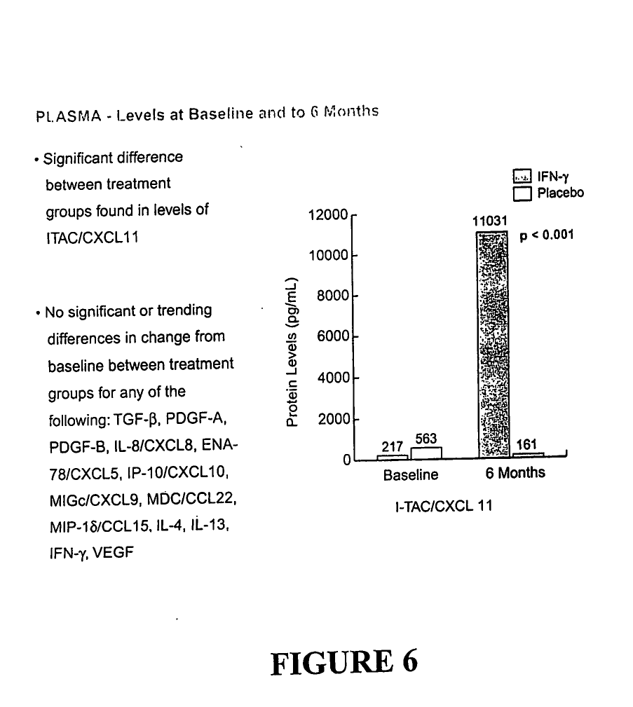

Data collected and analyzed in this study are shown in the FIGS. 2-6 and in the tables below:

| | IFN-γ 1b | Placebo |
|---|---|---|
| Baseline Clinical Characteristics | (n = 17) | (n = 15) |
| Prednisone (1-10 mg) (no. patients (%)) | 17 (100%) | 15 (100%) |
| Supplemental O₂ use (no. patients (%)) | 3 (18%) | 3 (20%) |
| Pa—O₂ (mm Hg, mean) | 78 mm Hg | 76 mm Hg |
| Aa-gradient (mmHg, mean) | 20 mm Hg | 21 mm Hg |
| FEV₁ (% predicted, mean) | 73% | 74% |
| FVC (% predicted, mean) | 67% | 68% |
| DL_{CO} (% predicted, mean) | 44% | 40% |
| Distance on 6MWT (meters, mean)^{a} | 423 m | 340 m |

^{a}p value = 0.035, Wilcoxon Rank Sum Test

| | IFN-γ 1b | Placebo |
|---|---|---|
| Baseline FVC | (n = 17) | (n = 15) |
| Mean ± SD (liters) | 2.7 ± 0.8 | 2.5 ± 0.7 |
| Mean ± SD (% of predicted) | 67 ± 12 | 68 ± 12 |
| Severity of Impairment | | |
| (% of predicted) | | |
| ≧70%- | 9 (53) | 4 (27) |
| ≧60%-≦70% | 2 (12) | 8 (53) |
| ≧50%-≦60% | 6 (35) | 3 (20) |

| | Lung TBBx | BAL Cell Pellet |
|---|---|---|
| | Patients with | Patients with |
| | mRNA present | mRNA present |
| Baseline Biomarkers | (% of total tested) | (% of total tested) |
| TGF-β | 28 (100) | 31 (100) |
| CTGF | 30 (100) | 29 (100) |
| Type I Procollagen | 27 (90) | 25 (100) |
| Type III Procollagen | 27 (96) | 28 (100) |
| Elastin | 28 (100) | 17 (100) |
| PDGF-A | 30 (100) | 29 (100) |
| PDGF-B | 29 (97) | 29 (100) |
| PDGF-D | 28 (93) | 30 (100) |
| IL-8/CXCL8 | 28 (100) | 31 (100) |
| ENA-78/CXCL5 | 29 (97) | 31 (100) |
| IP-10/CXCL10 | 26 (87) | 31 (100) |
| MIGc/CXCL9 | 28 (97) | 31 (100) |
| I-TAC/CXCL11 | 24 (86) | 31 (100) |
| MDC/CCL22 | 27 (90) | 29 (100) |
| MIP-1δ/CCL15 | 30 (100) | 31 (100) |
| IL-4 | 9 (30) | 4 (100) |
| IL-13 | 11 (37) | 6 (100) |
| IFN-γ | 23 (89) | 27 (100) |
| SMAD-7 | 27 (90) | 28 (100) |

SUMMARY OF SAFETY EVENTS [no. (%)]

| | IFN-γ 1b | Placebo |
|---|---|---|
| Patient Group | (n = 17) | (n = 15) |
| Total patients with any AE | 16 (94) | 13 (87) |
| Any serious AE | 1 (6) | 6 (40) |
| Dies | 0 (0) | 1 (7) |
| Discontinued treatment due to AE | 0 (0) | 1 (7) |

Treatment-Emergent AEs (%) Occurring in >15% of Patients

| | IFN-γ 1b | | Placebo |
|---|---|---|---|
| Event | (n = 17) | > or < | (n = 15) |
| Headache | 8 (47) | > | 3 (20) |
| Fever | 6 (35) | > | 1 (7) |
| Fatigue | 6 (35) | > | 2 (13) |
| Insomnia | 4 (24) | > | 2 (13) |
| Cough | 4 (24) | > | 0 (0) |
| Influenza-like illness | 3 (18) | < | 3 (20) |
| Myalgia | 3 (18) | > | 1 (7) |
| Rigors | 3 (18) | > | 1 (7) |
| Dyspnea | 3 (18) | > | 2 (13) |
| Upper respiratory infection | 2 (12) | < | 4 (27) |
| Pneumonia | 0 (0) | < | 3 (20) |

Serious Adverse Events

| | IFN-γ 1b | Placebo |
|---|---|---|
| Event | (n = 17) | (n = 15) |
| Total patients with any AE | 1 (6) | 6 (40) |
| Coronary artery occlusion | 0 | 1 |
| Chest pain | 0 | 1 |
| Malaise | 0 | 1 |
| Pyrexia | 0 | 1 |
| Pneumonia | 0 | 1 |
| Dehydration | 0 | 1 |
| Adenocarcinoma/Lung | 0 | 1 |
| Dyspnea, exacerbated | 0 | 1 |
| Dyspnea; NOS^{a} | 0 | 1 |
| Hypoxia | 0 | 1 |
| Pneumothorax | 0 | 1 |
| Surgery (left shoulder repair) | 1 | 0 |

^{a}Not otherwise specified

PRIMARY ENDPOINTS: RNA Transcription
Transbronchial Biopsy - mEDIAN mRNA Relative Expression
Change from Baseline to 6 Months

| mRNA (pg/mL) | IFN-γ 1b | Placebo | p Value |
|---|---|---|---|
| TGF-β | 1.30 (n = 17) | 1.42 (n = 9) | 0.811^{a} |
| CTGF | 1.25 (n = 17) | 0.62 (n = 11) | 0.433^{a} |

^{a}ANCOVA with classification effect for treatment and baseline as a covariate

| Patients with Changes in IL-4^{a} | IFN-γ 1b | Placebo |
|---|---|---|
| Decrease, no. (%) | 6 (35 [symbol] | 2 (18) |
| No change, no. (%) | 10 (59 [symbol] | 4 (36) |
| Increase, no. (%) | 1 (6 [symbol] | 5 (46) |

^{a}p value = 0.049, Wilcox rank sum test

SECONDARY ENDPOINT
BAL TOTAL CELLS - Alveolar macrophages, lymphocytes, and
neutrophils (mean in 1000 cells/mL) at Baseline and Change
from Baseline at 6 Months (Week 23-24)

| | IFN-γ 1b | Placebo | |
|---|---|---|---|
| Total Cells | (n = 17) | (n = 15) | p value^{a} |
| Baseline | 155 | 247 | |
| Change from Baseline^{b} | 17 | 22 | 0.315 |

Alveolar macrophages

| | | |
|---|---|---|
| Baseline | 116 | 168 |
| Change from Baseline^{b} | 32 | 28 |

Lymphocytes

| | | |
|---|---|---|
| Baseline | 17 | 36 |
| Change from Baseline^{b} | −7 | −3 |

Neutrophils

| | | |
|---|---|---|
| Baseline | 9 | 33 |
| Change from Baseline^{b} | 1 | −2 |

^{a}Wilcoxon rank sum test
^{b}At 6 Months

| | IFN-γ 1b | Placebo | |
|---|---|---|---|
| Endpoint | (n = 17) | (n = 15) | p value |
| FVC (adjusted mean change, | −0.036 | −0.096 | 0.435^{a} |
| liters) | | | |
| A-a gradient (adjusted mean | −0.272 | 5.965 | 0.054^{a} |
| change, mmHg) | | | |
| DLco (% pred, mean change) | −3.4 | −3.8 | 0.881^{a} |
| Dyspnea (MRC) (mean change) | 0.13 | 0.29 | 0.512^{b} |
| Dyspnea (TDI) (mean) | 0.2 | −1.4 | 0.216^{b} |
| Dyspnea (UCSD SOBQ) (mean change) | 6.2 | 8.3 | 0.573^{a} |
| Oxygen utilization (% no use) | 63% | 43% | 0.149^{b} |
| 6-MWT (adj mean change, meters) | −7.8 | −4.6 | 0.942^{a} |

^{a}ANCOVA with classification effect for treatment and baseline value include as a covariate
^{b}Wilcoxon rank sum test

### Example 3

### I-Tac/Cxcl11 Attenuates Bleomycin Induced Pulmonary Fibrosis

To determine if interferon-gamma- upregulated CXC chemokines, such as I-TAC/CXCL11 can attenuate clincial features of pulmonary fibrosis, mice were treated with I-TAC/CXCL11 in a bleomycine-induced pulmonary fibrosis model.

Mice (6-8 weeks old) were treated with intratracheal bleomycin (Blenoxane, Bristol Myers, Evansville, Ind., 0.15 U/kg) on day 0 as described in Keane et al., 1999, J. Immunol. 162:5511 and Smith et al., 1994 J. Immunol. 153:4704. Control animals received sterile saline. Briefly, mice were anesthetized with 250 μl of 12.5 μg/ml ketamine injected i.p., followed by intratracheal instillation of 0.025 U of bleomycin in 25 μl of sterile isotonic saline.

Mice were given daily injection I.M. of either I-TAC/CXCL11 (1 μg/day) or irrelevant protein as control from day 0 to day 12 post-bleomycin exposure. On day 12, mice were sacrificed for assay of soluble collegen in the lungs of treated and control mice.

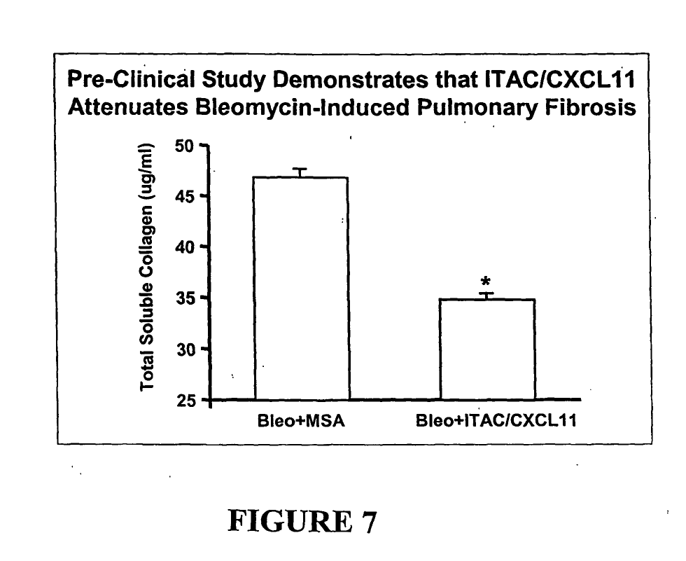

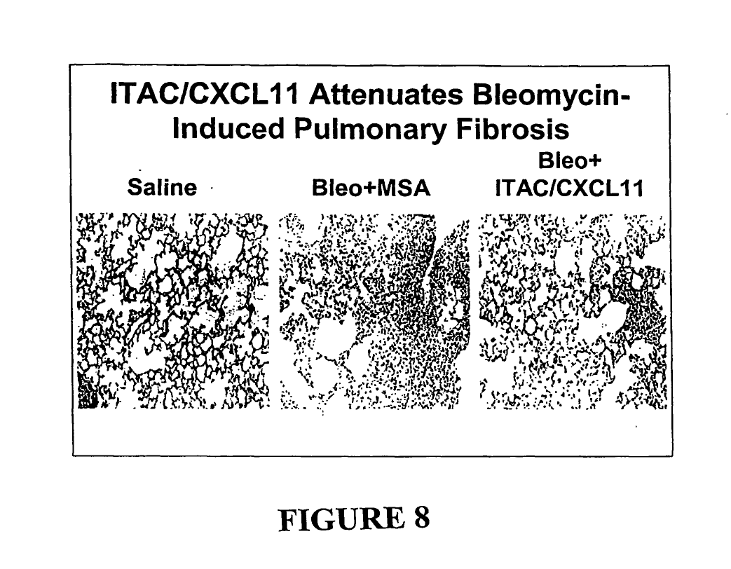

The data are shown in FIGS. 7 and 8, and demonstrate that systemic administration of I-TAC/CXCL11 significantly reduced bleomycin-induced pulmonary fibrosis, as determined by reduction in total soluble collagen deposition in the lung (FIG. 7) and preservation of the lung architecture (FIG. 8). These results demonstrate that the IFN-γ inducible CXC chemoline, I-TAC/CXCL11, inhibits fibrosis in a bleomycin-induced pulmonary fibrosis mouse model.

### Example 4

### ENA-78 Antagonist Reduces Aberrant Vascular Remodeling in IPF Lung

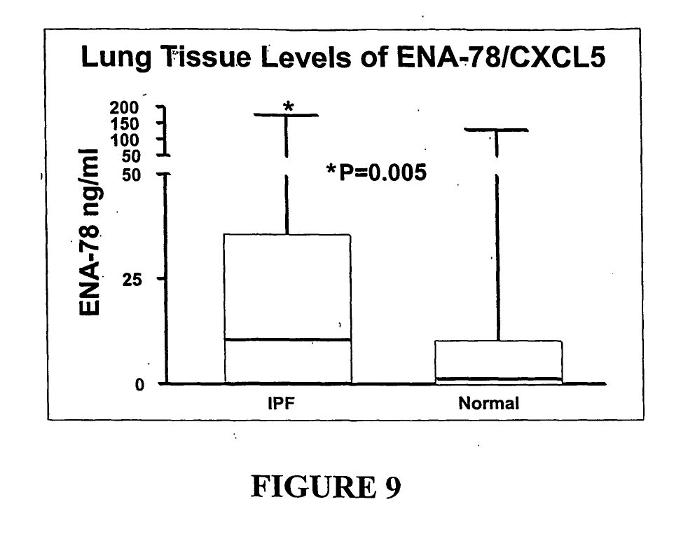

Lung tissue of IPF patients was evaluated for content of ENA-78/CXCL5 and compared with that of normal control lung tissue. As shown in FIG. 9, lung tissue from IPF patients contained elevated levels of ENA-78/CXCL5 as compared with normal tissue.

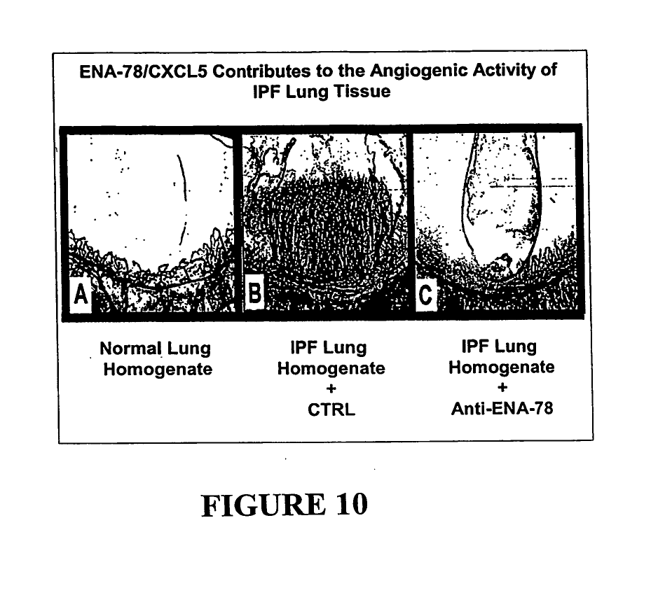

To evaluate the therapeutic effect of an antagonist of ENA-78/CXCL5 on IPF lung tissue, anti-ENA-78/CXCL5 antibody (10 μg) was added to IPF tissue homogenates (˜10 mg of total protein), then assessed for angiogenic activity in the rat cornea micropocket assay for angiogenesis. Data are shown in FIG. 10, and demonstrates that the IFN-γ-down-regulated CXC chemokine, ENA-78/CXCL5 is effective to reduce aberrant vascular modeling in IPF lung tissue.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1-42. (canceled)

43. A method of determining patient response to treatment with interferon gamma, comprising:
(a) analyzing expression of an IFN-gamma-regulated biomarker in an IFN-gamma-treated patient; and
(b) correlating the expression of one or more up-regulated biomarkers, or the expression of one or more down-regulated biomarkers, or both, relative to control expression, with patient response to interferon gamma treatment.

44. The method of claim 43, wherein said one or more up-regulated biomarkers comprise a CXCR3 ligand.

45. The method of claim 43, wherein said one or more up-regulated biomarkers comprise one or more biomarkers selected from the group consisting of I-TAC/CXCL11, IP-10/CXCL10, and MIG/CXCL9.

46. The method of claim 43, wherein said one or more down-regulated biomarkers comprise one or more biomarkers selected from the group consisting of ENA-78/CXCL5, IL-4, PDGFB, elastin, and procollagen.

47. The method of claim 43, wherein said up-regulated biomarker is I-TAC/CXCL11 and said down-regulated biomarker is ENA-78/CXCL5.

48. The method of claim 43, wherein said control expression is expression in a pre-IFN-gamma treatment patient sample.

49. The method of claim 43, wherein said correlating is between two or more samples obtained from the IFN-gamma treatment patient at different time periods post-treatment.

50. The method of claim 43, wherein mRNA expression is analyzed in transbronchial biopsy tissue.

51. The method of claim 43, wherein mRNA expression is analyzed in bronchoalveolar lavage cells.

52. The method of claim 43, wherein expression is analyzed by measuring protein in bronchoalveolar lavage fluid.

53. The method of claim 43, wherein expression is analyzed by measuring protein in blood or blood components.

54. The method of claim 43, wherein biomarker expression is analyzed about 2 hours to about 4 weeks after initiation of the interferon-gamma treatment.

55. The method of claim 43, wherein the patient suffers from idiopathic pulmonary fibrosis.

56. A method of treating a patient suffering from a pulmonary fibrotic disorder, comprising administering to the patient a therapeutic amount of I-TAC/CXCL11 and/or a therapeutic amount of an antagonist of ENA-78/CXCL5.

57. The method of claim 56, comprising co-administering to the patient a therapeutic amount of I-TAC/CXCL11 and a therapeutic amount of an antagonist of ENA-78/CXCL5.

58. The method of claim 57, wherein the amount of I-TAC/CXCL11 and the amount of the antagonist of ENA-78/CXCL5 antagonist are synergistically effective.

59. The method of claim 56, wherein the treatment is effective to reduce or avoid one or more selected from the group consisting of risk of death of the patient; dysregulated angiogenesis in the pulmonary vasculature of the patient; and morbidity or mortality due to infection in the patient.

60. The method of claim 56, further comprising co-administering to the patient a therapeutic amount of interferon gamma.

61. The method of claim 56, wherein the ENA-78/CXCL5 antagonist is an anti-ENA-78/CXCL5 antibody or antibody fragment.

62. The method of claim 61, wherein the anti-ENA-78/CXCL5 antibody or antibody fragment is a monoclonal antibody or fragment thereof.

63. The method of claim 56, further comprising co-administering to the patient a therapeutic amount of pirfenidone or a pirfenidone analog for the duration of therapy.

64. The method of claim 56, wherein the pulmonary fibrotic disorder is idiopathic pulmonary fibrosis.

65. The method of claim 64, wherein the patient exhibits a forced expiratory volume (FVC) of at least 55% of the patient's predicted normal FVC prior to treatment.

66. The method of claim 65, wherein the patient exhibits a FVC of at least 60% of the predicted normal FVC.

67. The method of claim 56, wherein treatment is maintained for the remainder of the patient's life.

68. The method of claim 43, further comprising the step of:
(c) maintaining, increasing or decreasing the dosage of interferon gamma in the treatment of the patient based on the results of step (b).

69. The method of claim 43, wherein expression is analyzed by measuring protein or mRNA in breath condensate.

70. Use of ITAC/CXC11 and/or an antagonist of ENA78/CXCL5, and optionally IFN-gamma in the manufacture of a medicament for treating a pulmonary fibrotic disorder.
